(19) United States
(12) Patent Application Publication
(10) Pub. No.: US 2025/0341495 A1
(43) Pub. Date: Nov. 6, 2025

(54) SURFACE-MICROMACHINED FABRY-PÉROT SENSORS AND ARRAY

(71) Applicant: The Texas A&M University System, College Station, TX (US)

(72) Inventors: Jun Zou, College Station, TX (US); Zhiyu Yan, College Station, TX (US); Zijie Zhao, College Station, TX (US); Cheng Fang, College Station, TX (US)

(21) Appl. No.: 18/862,423

(22) PCT Filed: May 2, 2023
(86) PCT No.: PCT/US2023/020734

§ 371 Date: Nov. 1, 2024

Related U.S. Application Data
(60) Provisional application No. 63/337,446, filed on May 2, 2022.

(51) Int. Cl. G01N 29/22; G01N 29/06; G01N 29/24; G01N 29/32

(52) U.S. Cl. CPC G01N 29/221; G01N 29/0672; G01N 29/32; G01N 29/2418; G01N 2291/106

(57) ABSTRACT

Highly uniform Fabry-Pérot-based optical ultrasound transducer arrays can be formed on a substrate by surface-micro-machining, using a sacrificial material layer to define the length of a Fabry-Pérot cavity formed between two partially optically reflective layers, and thereby set the optical resonance wavelength of the cavity. The transducer elements of the array can be interrogated in parallel with a laser operating near that resonance wavelength. Various techniques are available to tune the optical and acoustic properties of the transducer elements.

### CROSS-REFERENCE TO RELATED APPLICATION

This application claims priority to U.S. Provisional Patent Application No. 63/337,446, filed on May 2, 2022, the entire disclosure of which is incorporated herein by reference.

### STATEMENT OF GOVERNMENT RIGHTS

This invention was made with government support under Award Numbers NRI-1925037, CBET-2036134, CMMI-1852184, and ECCS-1809710 awarded by the National Science Foundation (NSF). The government has certain rights in this invention.

### BACKGROUND

High-sensitivity and high-density two-dimensional (2D) ultrasound transducer arrays are important for high-performance three-dimensional (3D) ultrasound and photoacoustic imaging and tomography, e.g., as used in medical applications such as brain imaging, drug discovery, or the detection of gene expression, to name just a few. In conventional ultrasound tomography, ultrasound waves are launched into a target, where they are scattered and reflected depending on the local optical scattering properties. In photoacoustic tomography (PAT), which has gained significant interest in recent years, short laser pulses are used to illuminate the target, and upon absorption of the incident laser pulses, ultrasound waves are generated inside the target with amplitudes closely related to the local optical absorption properties. In either case, the ultrasound transducer array measures ultrasound emanating from the target, which allows reconstructing a 3D image of optical scattering or absorption contrast based on the travel times of the ultrasound waves from their source points to the various points on the transducer array.

Currently, piezoelectric and capacitive ultrasound transducer arrays are most commonly used in ultrasound and photoacoustic imaging due to their ready availability. However, as these devices operate upon electric charge generation, their acoustic sensitivity decreases with the size of the transducer elements, which impedes their miniaturization and the formation of high-density 2D arrays. Also, the need for electrically interfacing a large number of elements makes the transducer array complex and costly. Further, because of practical limits on the number of available signal channels, simultaneous and parallel signal readout from all transducer elements is generally not feasible, and a multiplexing approach is instead often adopted for serial data acquisition from the array, which negatively affects imaging speed.

To achieve high-speed and high-resolution 3D ultrasound and photoacoustic imaging, a large-scale 2D array of ultrasound transducers that is capable of parallel data acquisition with high sensitivity is desirable.

### BRIEF DESCRIPTION OF THE DRAWINGS

This disclosure relates to ultrasound transducer arrays and sensor probes based on Fabry-Pérot sensors, as well as systems and methods for their optical interrogation and methods of their manufacture. The subject matter is described with reference to the following drawings, in which:

FIG. 1A is a schematic drawing of a Fabry-Pérot sensor in accordance with an embodiment;

FIG. 1B is a schematic graph of the resonance of the Fabry-Pérot sensor of FIG. 1A, illustrating its operating principle;

FIGS. 2A-2F are schematic drawings illustrating various stages of a method of manufacturing an ultrasound transducer array of Fabry-Pérot sensors, in accordance with an embodiment;

FIGS. 3A-3C are schematic drawings of Fabry-Pérot sensors with various added layers for optical resonance wavelength control in accordance with various embodiments;

FIG. 4A is a graph showing the optical reflection spectrum of an example ultrasound transducer element in accordance with an embodiment;

FIG. 4B is a map showing the optical resonance wavelength across a center region of an example ultrasound transducer array of Fabry-Pérot sensors in accordance with an embodiment;

FIG. 4C is a histogram showing the distribution of optical resonance wavelengths over the example ultrasound transducer array of FIG. 4B;

FIGS. 5A and 5B are graphs showing the acoustic frequency response spectra of example ultrasound transducer elements of different respective sizes, illustrating acoustic frequency response control in accordance with an embodiment;

FIGS. 5C-5F are graphs showing the acoustic frequency spectra of ultrasound signals measured with example ultrasound transducer elements having damping layers of different respective thicknesses, illustrating acoustic frequency response control in accordance with various embodiments;

FIGS. 6A and 6B are graphs showing example ultrasound signals received by a needle hydrophone and an example ultrasound transducer element in accordance with an embodiment, respectively;

FIG. 6C is a map showing noise equivalent pressure (NEP) values across a center region of an example ultrasound transducer array in accordance with an embodiment;

FIG. 7A is a graph showing the average optical resonance wavelength of a set of ultrasound transducer elements of an example array, in accordance with an embodiment, as a function of temperature;

FIG. 7B is a graph showing the average optical resonance wavelength of the set of ultrasound transducer elements of FIG. 7A immersed in water over a period of time;

FIGS. 8A and 8B are schematic drawings of ultrasound transducer array devices with and without integrated microlenses, respectively, in accordance with various embodiments;

FIGS. 9A and 9B are bottom views of microlens arrays with circular and square cross-sectional shapes, respectively, in accordance with various embodiments;

FIG. 10 is a graph of ultrasound signal waveforms measured with various example ultrasound transducer array devices with and without integrated microlenses in accordance with various embodiments;

FIG. 11 is a scatter plot of the focal length of example microlenses as illustrated schematically in FIG. 8B as a function of aspect ratio;

FIG. 12A is a schematic drawing of an ultrasound transducer array device with integrated optical fibers, in accordance with an embodiment;

FIG. 12B is a schematic drawing of a fiber-optic Fabry-Pérot sensor probe in accordance with an embodiment;

FIG. 12C is a schematic drawing of a curved ultrasound transducer array device constructed from fiber-optic Fabry-Pérot sensor probes in accordance with an embodiment;

FIG. 13 is a schematic diagram illustrating an example photoacoustic imaging system utilizing an ultrasound transducer array in accordance with an embodiment; and

FIG. 14 is a conceptual signal-flow diagram illustrating an aspect of the operation of the photoacoustic imaging system of FIG. 13.

### DESCRIPTION

Described herein are optically interrogated (or, in short, optical) ultrasound transducer arrays and sensor probes based on Fabry-Pérot sensors. Optical ultrasound transducers convert ultrasound signals into optical signals through optomechanical modulation, that is, the change of an optical quantity (such as a resonance wavelength) due to a change in a geometric or optical parameter of the transducer in response to ultrasound. Fabry-Pérot-based ultrasound transducers, in particular, are interferometric devices whose optical resonance wavelength changes as a result of variations in the Fabry-Pérot cavity length when ultrasound impinges on the transducer. Beneficially, the sensitivity of optical ultrasound transducers—unlike that of piezoelectric or capacitive transducers—is determined mainly by the intensity of the interrogating light and the modulation efficiency, and is independent of the lateral size of the ultrasound transducer elements, rendering it possible to maintain high sensitivity while reducing the transducer element size. Further, as between interferometric and refractometric optical ultrasound transducer, the former generally provide better sensitivity. Interferometric optical ultrasound transducers, as described herein, therefore lend themselves to implementing large, high-density, and yet high-sensitivity arrays. In addition, the optical ultrasound transducer elements of the array can, at least in principle, be read out in parallel without massive electrical wiring. Further, as compared with sensors utilizing microring resonators or Bragg gratings, which are lateral two-dimensional structures difficult to configure in dense arrays and generally requiring complex readout interfaces in plane with the transducer elements, Fabry-Pérot-based ultrasound transducer elements arrayed on a substrate are interrogated in a direction normal to the substrate, and as such conveniently accessible. For these reasons, Fabry-Pérot-based optical ultrasound transducer arrays are well-suited to high-speed and high-resolution 3D ultrasound and photoacoustic imaging.

To fully leverage the benefits principally provided by optically interrogated ultrasound transducer arrays, it is important that the arrays exhibit high uniformity in their optical and acoustic properties. Achieving such uniformity, however, is no trivial endeavor. For example, arrays of Fabry-Pérot-based sensor elements manufactured by bulk etching Fabry-Pérot cavities into a substrate and then bonding a flexible diaphragm to the etched substrate to create cavities bounded at the top by diaphragms that flex in response to ultrasound suffer from difficulties in precisely controlling the bulk etching depth. Similarly, ultrasound transducer arrays manually assembled from fiber-optically interrogated individual transducer elements are subject to imprecision in the distances between the optical fiber tips and diaphragms. Undesirable variations in cavity length across the array that result from this lack of control may necessitate continuous optical tuning of each transducer element, which is a tedious and serial process that would seriously limit data acquisition speed. In accordance herewith, therefore, a different approach to manufacturing an ultrasound transducer array of Fabry-Pérot sensors is taken.

In various embodiments, an optical ultrasound transducer array is surface-micromachined on a substrate by depositing a planar partially optically reflective layer (e.g., including a stack of sub-layers forming a distributed Bragg reflector) on the substrate, depositing a sacrificial layer on the planar partially optically reflective layer, patterning the sacrificial layer to form an array of islands of sacrificial material, depositing a second partially optically reflective layer over the array of islands on the first partially optically reflective layer, and then removing the sacrificial material to form an array of Fabry-Pérot cavities between the two partially optically reflective layers, and vacuum-sealing the cavities with a sealing layer. The Fabry-Pérot cavities are bounded at the bottom by the first, planar partially optically reflective layer and at the top and sides by respective flexible diaphragms and side walls formed by the second partially optically reflective layer. Together, the array of Fabry-Pérot cavities, whose cavity length (i.e., the size of the gap between the top diaphragm and the substrate) can be optically measured, and the respective flexible diaphragms, which bend responsive to ultrasound, form an array of optical ultrasound transducer elements. Beneficially, the thickness of the sacrificial layer, which directly determines the cavity length of cavities across the array, can be controlled to a high degree (e.g., within a few nanometers using standard thin-film deposition techniques), enabling manufacture of an array of Fabry-Pérot cavities that are highly uniform in their baseline cavity lengths (meaning their cavity lengths in the absence of impinging ultrasound) and, consequently, their optical resonance wavelength. High uniformity is achievable even at small transducer dimensions (e.g., of 200 μm or less). For example, in one embodiment, the uniformity of an array of optical ultrasound transducer elements having dimensions of less than 100 μm is characterized by 90% or more of the optical ultrasound transducer elements having optical resonance wavelengths within a range of only ±5 nm from the average optical resonance wavelength of the array. In addition to high uniformity, the described surface-micromachined optical ultrasound transducer arrays also benefit, in various embodiments, from high thermal and temporal stability.

Moreover, optical ultrasound transducer arrays in accordance herewith are amenable to customization, by design and/or tuning post-manufacture, of their optical and/or acoustic properties. The mechanical (or, synonymously, acoustic) frequency response of the acoustic sensor can be controlled via the lateral size of the transducer elements, which determines the center frequency of the transducer's response spectrum, and the thickness of material acting as the flexible diaphragm, which determines the acoustic bandwidth. In various embodiments, a damping layer, e.g., of a polymer or elastomer material, is deposited on top of the sealing layer, or directly on top of the upper partially reflective layer, to increase the acoustic bandwidth. The thicker the damping layer, the larger the damping effect and the corresponding increase in bandwidth will be. Alternatively or additionally, the pressure inside the sealed cavity can be increased during manufacture to achieve damping. In some embodiments, the acoustic bandwidth is greater than 80%, or even around or greater than 100%, of the center frequency of the acoustic frequency response. Further, to avoid spatial aliasing and grating lobes in a directivity profile of the ultrasound transducer, the pitch of the array of optical ultrasound transducer elements is chosen, in accordance with some embodiments, to be less than half of the acoustic wavelength at the center frequency of the acoustic frequency response spectrum.

The optical resonance frequency of the transducer array is determined primarily by the thickness of the sacrificial layer deposited during manufacture. However, fine-tuning of the optical resonance frequency is possible post-sealing of the cavity, e.g., passively by adjusting the total thickness of the diaphragm (e.g., by thinning down or adding to the sealing and/or damping layers), or actively by patterning electrodes for the application of a voltage across the cavity or by adding a coating to form a bimorph structure between the upper reflective layer and the coating for thermally induced bending of the diaphragm.

The disclosed method of manufacturing Fabry-Pérot-based optical ultrasound transducer arrays, in providing control over the optical resonance frequency of the array, facilitates choosing the resonance wavelength depending on the operating wavelength of the laser or other components of the system used to optically interrogate the transducer array, rather than the other way round. Thus, the optical ultrasound transducer array can be designed, in accordance with one embodiment, for a resonance wavelength in the vicinity of 532 nm, which enables use of a Nd:YAG laser to provide interrogation light pulses for the pulsed illumination of the transducer array and of a conventional charged-coupled device (CCD) or complementary metal-oxide semiconductor (CMOS) camera to capture the reflected light. Beneficially, Nd:YAG lasers can achieve very short pulses (e.g., less than 10 ns in length) at pulse energies sufficient to obtain a good signal level (e.g., 0.1 mJ per pulse), and—like cameras suitable for detection around 532 nm—are commercially available at low cost, as compared with pulsed lasers and cameras operating, e.g., in the near-IR range.

In accordance with various embodiment, the optical ultrasound transducer array and interrogation system are configured for illumination of the array from the back, i.e., through the substrate (which may be made of glass or some other material transparent to light at the operating wavelength) and in a direction opposite to the direction in which ultrasound impinges on the transducer elements. The optical interrogation may be achieved with an expanded and collimated beam of light, e.g., transmitted via free space until it hits the back surface of the substrate. Alternatively, an optical fiber bundle (e.g., substantially matching the dimensions of the array) may deliver the interrogating light, e.g., up to the back surface of the substrate, and capture the reflected light to create an image of the ultrasound transducer array. Use of a fiber bundle in lieu of free-space transmission generally reduces constraints imposed on the relative spatial arrangement between the interrogating laser and the transducer array, and is as such suitable for ultrasound imaging probes intended for use inside the body or other tight spaces. Both a free-space collimated beam and an optical fiber bundle allow for the parallel read-out of multiple transducer elements or even the entire array. However, with a low filling factor of the transducer elements, the blank areas surrounding each element would result in power losses. In addition, because the light illuminating each transducer element is not focused, the efficiency of the optomechanical modulation is compromised. To avoid the degradation of the acoustic sensitivity and responsivity of the transducer elements that is incidental to these effects, an optical interrogation array is integrated with the ultrasound transducer array to direct light through the back surface of the substrate into and collect reflected light from the individual optical ultrasound transducer elements of the array. In various embodiments, this transducer array can take the form of, alternatively, a microlens array created on the back surface of a transparent substrate or an array of optical fibers inserted through the back surface of the substrate, each microlens or optical fiber being aligned with one of the ultrasound transducer elements.

In some embodiments, the array of Fabry-Pérot sensors created on a substrate, e.g., with integrated optical fibers for interrogation, is diced to create individual (fiber-optic) Fabry-Pérot-based sensor probes. These sensor probes can, in turn, be affixed to a curved surface, e.g., of a support structure, to form a curved ultrasound transducer array. Alternatively (or additionally), various functional coatings may be applied to the top diaphragm of the Fabry-Pérot cavities to create sensor probes of different types, such as, alternatively to ultrasound probes, moisture, temperature, or chemical sensors.

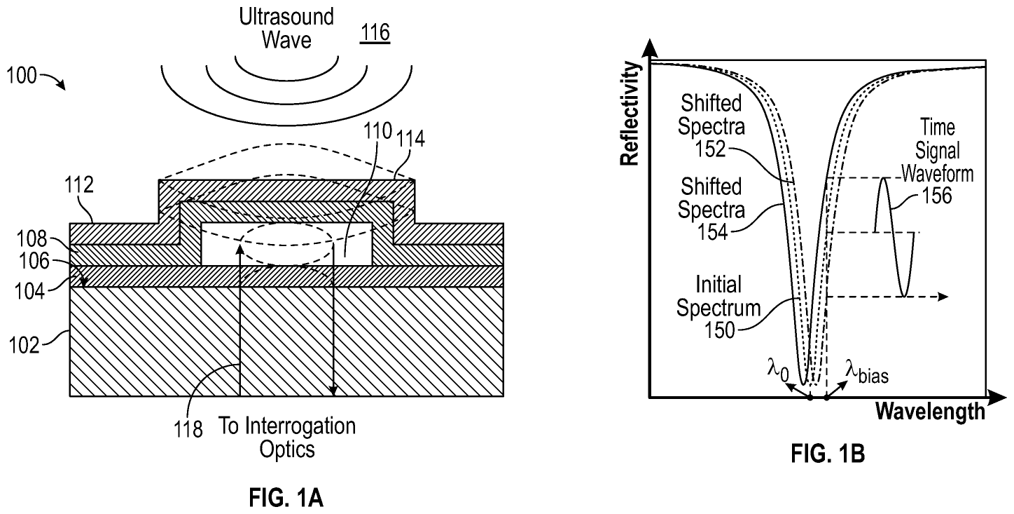

Following this general overview of various aspects of the disclosed subject matter, specific embodiments and examples will now be described in more detail with reference to the accompanying drawings. While the following, like the foregoing, description contains numerous references to ultrasound transducers, it is to be understood that, as alluded to above, the same sensor structures, enhanced with suitable functional coatings, can also provide sensing functionality other than or beyond ultrasound detection. Also, in general, the disclosed transducer structures are not limited to a particular sound frequency range, and the term “ultrasound” is, accordingly, substitutable by the more general term “acoustic.” That said, the detection of ultrasound specifically is an important application of the disclosed Fabry-Pérot-based sensor arrays, and indeed motivates the transducer array structure disclosed herein. Turning now to the drawings, FIG. 1A is a schematic drawing of a

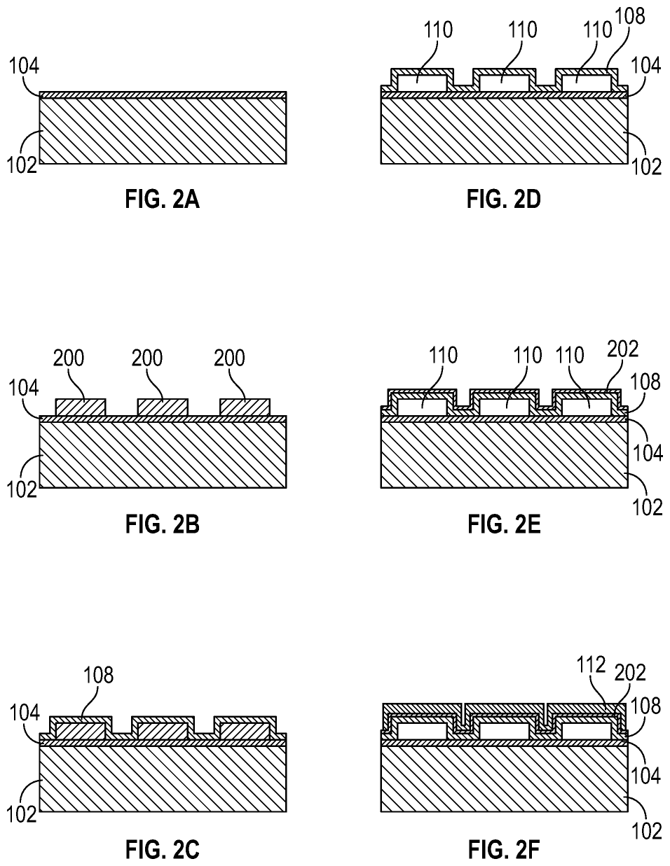

Fabry-Pérot sensor 100 in accordance with an embodiment. The sensor 100 is formed on a substrate 102, and includes a planar partially optically reflective layer 104 (herein also “first partially optically reflective layer” or “bottom partially reflective optical layer”) disposed on a front surface 106 of the substrate 102, and another partially optically reflective layer 108 (herein also “second partially optically reflective layer” or “top partially optically reflective layer”) disposed over the first partially optically reflective layer 104 such that a cavity 110 is defined between the first and second partially reflective layers 104, 108. The cavity 110 is bounded at the bottom by the first partially optically reflective layer 104 and at the top and sides by the second partially optically reflective layer 108. (The term “disposed over,” thus, is not intended to imply that the second layer 108 has to be in physical contact with the first layer 104 across its entire area.) The cavity 110 is sealed, e.g., with an additional sealing layer (not shown in FIG. 1A) disposed over the second partially optically reflective layer 108 to close what would otherwise remain as a manufacturing-related gap in the enclosure of the cavity 110 (as explained with reference to FIGS. 2A-2F). Optionally, a damping layer 112 may be disposed over or above the second partially optically reflective layer 108. At the top of the cavity 110, the second partially optically reflective layer 108 and any layers (e.g., 112) disposed thereon form a flexible diaphragm 114 that, as indicated with dotted lines, can bend up and down in response to ultrasound waves 116 impinging on the diaphragm 114. The acoustic response of the diaphragm 114 depends on the pressure inside the cavity 110 and the materials and thickness of the diaphragm 114.

The first and second partially optically reflective layers 104, 108 are, in some embodiments, distributed Bragg reflectors (DBRs) each formed by a stack of sub-layers alternating between two materials, such as between an oxide (e.g., silicon oxide) and a nitride (e.g., silicon nitride). Such DBRs reflect light across a reflection band (e.g., 100-200 nm wide) that, in frequency, is centered around a center frequency whose associated center wavelength is equal to four times the optical length (thickness times refractive index) of each sub-layer, or twice the optical length of each pair of sub-layers, of the stack. The DBRs of the two partially reflective layers 104, 108 are generally configured to share the same reflection band (or at least substantially overlap in their reflection bands). Alternatively to being DBRs, the first and second partially optically reflective layers 104, 108 may be, e.g., metal thin films having a wide reflection band.

As a result of the partial optical reflectivity of the layers 104, 108, the cavity 110 constitutes a Fabry-Pérot cavity that can be interferometrically interrogated by coherent light having a wavelength within the reflection band of the layers 104, 108. As indicated in FIG. 1A by the arrows 118, light illuminating the Fabry-Pérot sensor 100 through the substrate 102 and impinging on the bottom partially optically reflective layer 104 is reflected in part and transmitted in part into the cavity 110. Light impinging inside the cavity 110 on the top partially optically reflective layer 108 is, again, partially reflected, and a portion of the reflected light is transmitted through the bottom layer 104, whereas another portion is reflected back towards the top layer 108. If the wavelength of the light is twice the gap between the top and bottom partially optically reflective layers 104, 108 (called the “cavity length” of the Fabry-Pérot cavity 110), the Fabry-Pérot cavity is at resonance and its reflectivity at a minimum. Flexing of the diaphragm 114 formed by the top layer 108 (and any layers deposited thereon), e.g., due to impinging ultrasound, slightly changes the cavity 110, and thus the resonance wavelength. By configuring the cavity 110 and its bonding top and bottom partially optically reflective layers 104, 108 such that the resonance wavelength of the cavity (twice the cavity length) is within the reflection band of the layers 104, 108, these cavity length changes can be optically measured via the shift in the resonance wavelength. The Fabry-Pérot cavity 110 and its bounding top flexible diaphragm 114 together thus form an optical ultrasound transducer elements.

FIG. 1B is a schematic graph of the resonance of the Fabry-Pérot sensor of FIG. 1A, illustrating its operating principle. The initial reflection spectrum 150 has its lowest reflectivity, called its optical resonance wavelength, at λ₀. As the flexible diaphragm 114 vibrates due to impinging ultrasound waves, the reflection spectrum shifts (e.g., to spectra 152, 154), and with it the optical resonance wavelength. By interrogating the Fabry-Pérot sensor at a wavelength λ_{bias} that is located between the optical resonance wavelength λ₀ and a wavelength along the resonance dip giving high reflectivity, i.e., somewhat offset from λ₀, this shift in the optical resonance can be translated into a variation in the optical power of the reflected light, as indicated by curve 156. To maximize the linearity between the optical signal and the acoustic pressure, the interrogation wavelength λ_{bias} is preferably chosen around the midpoint between the lowest and highest reflectivity.

FIGS. 2A-2E are schematic drawings illustrating various stages of a method of manufacturing an ultrasound transducer array of Fabry-Pérot sensors (such as sensor 100 depicted in FIG. 1A), in accordance with an embodiment.

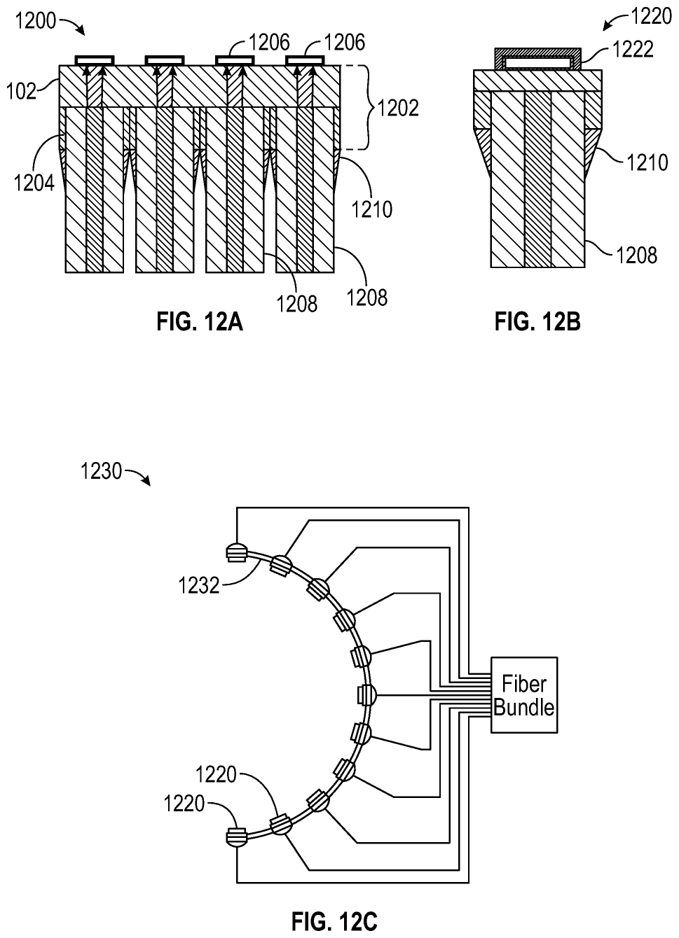

The method may employ standard surface micromachining techniques, such as, e.g., thin-film deposition techniques such as chemical vapor deposition (CVD) or plasma-enhanced CVD. The array is created on a substrate 102 that may be made of glass (or some other material that is transparent at least at the interrogation wavelength), e.g., having a thickness of a few or several hundreds of micrometers (e.g., 500 μm). Alternatively, as illustrated in FIGS. 12A-12C below, a multi-layer substrate including a top glass layer may be used. A planar first partially optically reflective layer 104 is deposited on a front surface of the substrate 102 (FIG. 2A). The layer 104 may, for example, be a DBR consisting of oxide and nitride (e.g., SiO₂ and SiNₓ) layers, with thickness and refractive index of the oxide/nitride films being controlled to obtain a desirable reflectivity of the DBR. For example, in one embodiment, the deposition of the oxide and nitride layers on the substrate 102 is preceded by deposition of individual dummy oxide and nitride layers (e.g., on a different substrate) and measurement of the refractive indexes of the dummy layers; the thicknesses of the oxide and nitride layers forming the DBR are then selected based on the measured refractive indexes such that the oxide and nitride layers each have an optical length equal to one quarter of a targeted center wavelength of the reflection band of the distributed Bragg reflector.

Following deposition of the first, planar partially optically reflective layer, a sacrificial layer, e.g., a zinc oxide (ZnO) film, is deposited on the first partially optically reflective layer 104, and patterned (e.g., photolithographically, as known in the art) to form islands 200 of sacrificial material (FIG. 2B). A second partially optically reflective layer 108, such as a second DBR configured the same as the first, is then deposited over the array of sacrificial-material islands 200 on the first partially optically reflective layer 104 (FIG. 2C). Next, the sacrificial-material islands 200 are etched to create an array of Fabry-Pérot cavities 110 defined between the first and second partially optically reflective layers 104, 108 (FIG. 2D). A sealing layer 202 is then deposited over the second partially optically reflective layer 108 to close openings therein through which the etchant for removing the sacrificial material was applied, and thereby to vacuum-seal the Fabry-Pérot cavities 110 (FIG. 2E). The sealing layer may be, e.g., a metal, oxide, or polymer layer. In various embodiments, a low-temperature oxide (LTO) (e.g., a silicon oxide deposited at the low temperature of between 400 and 500 degrees Celsius) is deposited as the sealing layer because of its good sealing properties, which allow maintaining a vacuum inside the cavities 110 for a long time. An additional layer of material may be deposited over the sealing layer 202 to serve as an acoustic damping layer 112 (FIG. 2F). The damping layer 112 may, for instance, be made of a polymer (such as, e.g., parylene), an elastomer (such as, e.g., PDMS), or an epoxy, and may have a thickness in the range from 1 μm to 50 μm. In addition to broadening the acoustic frequency response of the transducer elements via damping, the damping layer can also serve to protect the array from abrasion and moisture. While separate sealing and damping layers 202, 112 are shown, it is in principle also possible that a single layer of material fulfills both sealing and damping functions.

The manufacturing method illustrated in FIGS. 2A-2F allows controlling the cavity length, and thus the optical resonance wavelength, via the thickness of the deposited sacrificial layer. However, while the sacrificial layer thickness is equal to the cavity length when the top diaphragm is in a neutral position, removal of the sacrificial material tends to cause the diaphragm to buckle up slightly, and vacuum-sealing the cavity tends to cause the diaphragm to buckle downward slightly under atmosphere. Different methods can be used to precisely control the cavity length and optical resonance wavelength by tuning after cavity sealing.

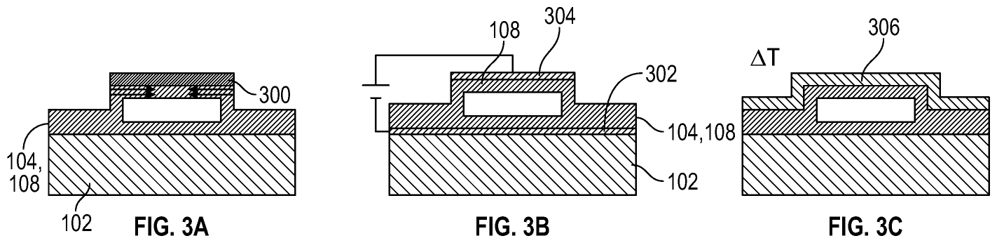

FIGS. 3A-3C are schematic drawings of Fabry-Pérot sensors with various added layers for optical resonance wavelength control in accordance with various embodiments. FIG. 3A depicts a way to passively tune the cavity length by adjusting the stress and thickness of the diaphragm via coatings 300 applied on top of the diaphragm created by the top partially optically reflective layer 108. For example, more sealing or damping material may be deposited to enlarge the cavity length, or the sealing layer may be thinned down (e.g., LTO may be wet-etched with hydrofluoric acid (HF)) to shrink the cavity length. The cavity length can also be actively controlled. For instance, FIG. 3B depicts a sensor with two transparent electrodes 302, 304 deposited and patterned beneath the bottom partially optically reflective layer 104 and above the top partially optically reflective layer 108 (e.g., on top of the sealing layer), which allows tuning the cavity length via electrostatic force by application of a voltage between the electrodes 302, 304. Yet another approach, illustrated in FIG. 3C, is to control the cavity length utilizing the bimorph effect. For this purpose, an additional coating 306, e.g., of metal, is applied above the top partially optically reflective layer (e.g., on top of the sealing layer and below any damping layer) to form a bimorph structure. The additional coating 306 has a thermal coefficient of expansion that differs substantially (e.g., by at least 10%) from that of the top partially optically reflective layer, such that temperature changes result in buckling of the bimorph structure, allowing the cavity length to be actively adjusted via the temperature.

As noted, a benefit of the manufacturing method illustrated above with reference to FIGS. 2A-2F is the ability to create ultrasound transducer arrays with very uniform optical resonance wavelength. For example, with an array of transducer elements each having dimensions of less than 100 μm, a level of uniformity characterized by at least 90% of the optical ultrasound transducer elements having associated optical resonance wavelengths deviating by no more than 5 nm from an average optical resonance wavelength of the array can be achieved in some embodiments.

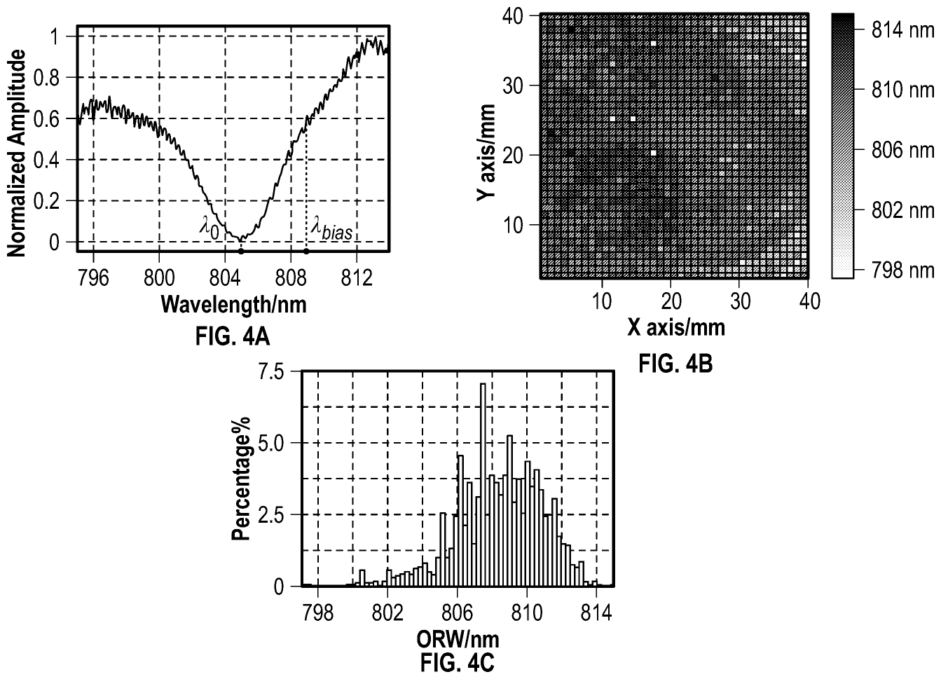

Evidence of such optical uniformity is provided in FIGS. 4A-4C for an example 5 cm×5 cm ultrasound transducer array of 350×350 elements, each 70 μm in diameter, arranged at a pitch of 140 μm. To characterize the optical reflection spectrum of the transducer elements, a 765-815-nm continuous-wave tunable laser was used to interrogate the array, while a halogen lamp served to illuminate the measured element. To simulate its actual working conditions, the array was immersed in water with its backside facing toward the incident light beam. The laser and the collimated white light beams were combined with a dichroic mirror and focused onto the center region of an element through the glass substrate by a 10× objective lens. Reflected light from the element was coupled into a single mode (SM) fiber coupler and received by a photodetector. Part of the reflected white light was split by a beam splitter and projected onto a CCD camera for monitoring the location of the element under testing. The output of the photodetector was amplified and recorded by a data acquisition card (DAQ) synchronized by a trigger signal from the tunable laser. The recorded time-domain signal during one sweeping cycle of the tunable laser was converted into the reflection spectrum. To characterize the optical uniformity, the interrogation laser spot was scanned over the array with a motorized two-axis stage with scanning steps of 0.98 mm×0.98 mm (every 7 elements) and a scanning range of 40×40 steps (˜4 cm×4 cm).

FIG. 4A is a graph showing the optical reflection spectrum of an example ultrasound transducer element of the array. As can be seen, the resonance wavelength in this example is at around 805 nm. FIG. 4B is a map showing the optical resonance wavelength across a center region of the array, and FIG. 4C is a histogram showing the distribution of optical resonance wavelengths over the array. In this example, more than 94% of the elements (excluding those at the corners) have an optical resonance wavelength between 802 and 812 nm.

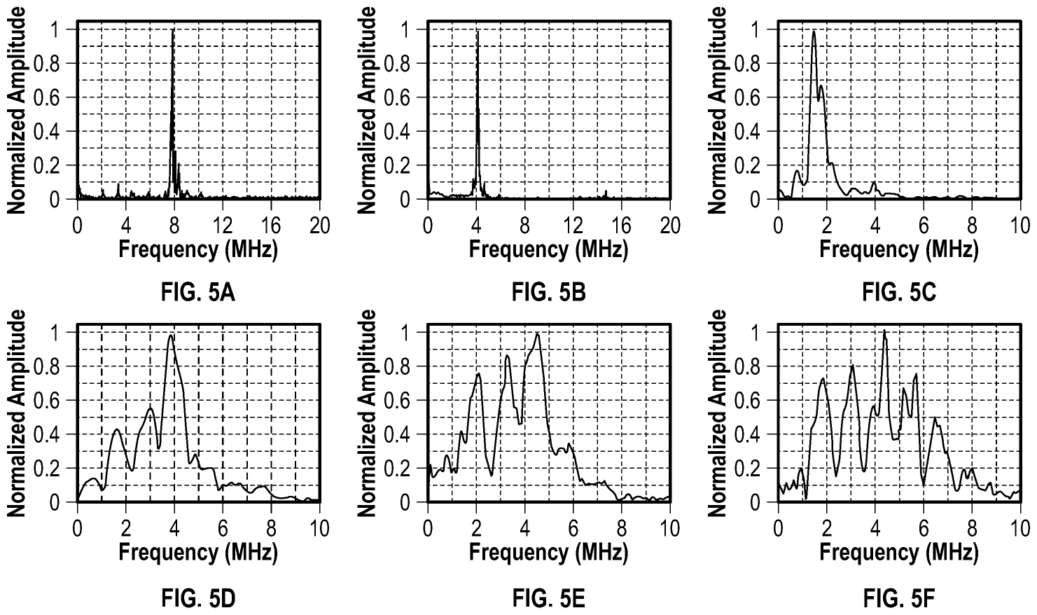

In addition to providing control over the optical resonance wavelength, optical ultrasound transducer elements as described herein can be designed for a desired acoustic frequency response. Both the center frequency of the response and the acoustic bandwidth affect the imaging performance of the ultrasound transducer, and are desirably carefully controlled. The center frequency is determined by the lateral size of the transducer elements (more specifically, the diameter or lateral dimensions of the flexible diaphragm of each element). The acoustic bandwidth is determined in part by damping, which can be enhanced with a damping layer, e.g., of parylene or another polymer coating. The thicker the damping layer, the larger the damping effect will be. (FIGS. 5A-5F described below illustrate these relationships.) Accordingly, in various embodiments, the dimensions of the ultrasound transducer elements and the thickness of the damping layer are selected at least in part based on a target center frequency and target bandwidth, respectively, of the acoustic frequency response spectrum. In some embodiments, the acoustic frequency spectrum of the optical ultrasound transducer elements is characterized by a bandwidth greater than 80%, or of about or greater than 100%, of a center frequency of the acoustic frequency spectrum. It is noted, however, that increased damping layer thickness will reduce the responsivity of the transducer element, representing a trade-off between responsivity and bandwidth that calls for optimization for satisfactory sensor performance given the circumstances of the sensing application.

FIGS. 5A and 5B are graphs showing the acoustic frequency response spectra of example ultrasound transducer elements of different sizes: 50 μm and 80 μm, respectively. The spectra were evaluated using a laser vibrometer. As can be seen, by increasing the later size from 50 μm to 80 μm, the center frequency is decreased from 7.9 MHz to 4.0 MHz. FIGS. 5C-5F are graphs showing the acoustic frequency spectra of ultrasound signals measured with example ultrasound transducer elements having parylene-c damping layers of different respective thicknesses: 0 μm (no damping layer), 4 μm, 9 μm, and 23 μm, respectively. The measured ultrasound signals were pulses transmitted by a 25 MHz focused ultrasound transducer. As can be seen in FIGS. 5C-5F, both center frequency and bandwidth of the measured signals were increased by enlarging the damping layer thickness.

Alternatively to increasing the damping layer thickness, one way to effectively control the acoustic bandwidth of the frequency response of an ultrasound transducer element is via the pressure inside the sealed cavity. Trapped air damps the flexible diaphragm, widening the bandwidth. The pressure inside the cavity can be controlled, in accordance with one embodiment, by first patterning polymer material at (but without blocking) the etching via used to remove the sacrificial material, and then increasing the temperature of the transducer element inside a vacuum oven until the polymer patterns are reflowed, sealing the cavity. The pressure inside the sealed cavity is determined by the ambient pressure during sealing, which can be adjusted by the vacuum oven. Thus, bandwidth control can be achieved by vacuum-sealing the Fabry-Pérot cavities of the ultrasound transducer array inside a vacuum oven at an ambient pressure selected based on a target internal pressure inside the cavities corresponding to a target bandwidth of the acoustic frequency spectrum.

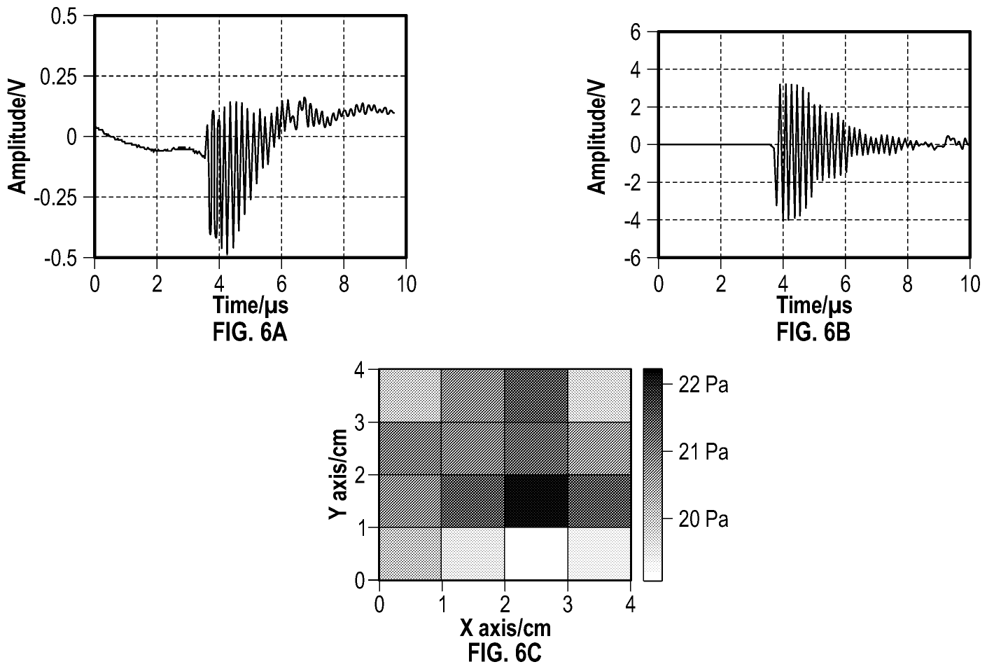

The sensitivity of an optical interferometric ultrasound transducer is determined by both the optical and the mechanical sensitivity. The optical sensitivity is mainly affected by the finesse of the interferometer, and the mechanical sensitivity mostly depends on the stiffness and acoustic resonance behavior of the top diaphragm. The overall sensitivity of the transducer array and the optical read-out setup can be evaluated in terms of the NEP, which is defined as the detected acoustic pressure level when the signal to noise ratio (SNR) reaches one in the low-frequency limit (when acoustic wavelength is much larger than cavity length of the Fabry-Pérot cavity): NEP=P/SNR, where P is the acoustic pressure (Pa) received by the sensor. FIGS. 6A-6C show the NEP characterization of the example transducer array and experimental setup described with reference to FIGS. 4A-4C. The transducer array was placed onto a holder with the device side facing down toward a lead zirconate titanate (PZT) plate having a thickness of 0.4 mm and a thickness-mode resonance frequency of 5 MHz that served as the acoustic source.

FIGS. 6A and 6B are graphs showing example ultrasound signals resulting from acoustic pressure generated by the PZT plate, measured by a needle hydrophone in a center region of the transducer array and by one of the ultrasound transducer elements, respectively. For the needle hydrophone measurement, the averaged signal from five different locations was 575 m V. With a hydrophone responsivity of 60 mV/MPa and 50-dB amplification, the acoustic pressure was estimated to be 30.2 kPa. For the ultrasound transducer array, the averaged signal amplitude of ultrasound signals measured with twenty-five elements within a 4 cm×4 cm center region of the array was 7.3 V.

FIG. 6C is a map showing NEP values across the center region of the ultrasound transducer array. The peak-to-peak noise amplitude was measured at the time range between trigger and ultrasound signal pulse; the noise originated mainly from the continuous laser, photo detector, and the amplifier. The noise amplitude was determined to be 41.6 mV without signal averaging and 5.0 mV with averaging over sixteen signals, respectively. The averaged NEP was calculated to be 172.5 Pa without signal averaging and 20.7 Pa with signal averaging (over sixteen signals) over a bandwidth of 10 MHz. As can be seen in FIG. 6C, the NEP (with signal averaging) of the twenty-five elements ranged between 19.5 Pa and 22.5 Pa, which means that the sensitivity of the elements is quite uniform across the entire array. The NEP of the optical ultrasound transducer array is also much lower than those of piezoelectric needle hydrophones (˜6 KPa).

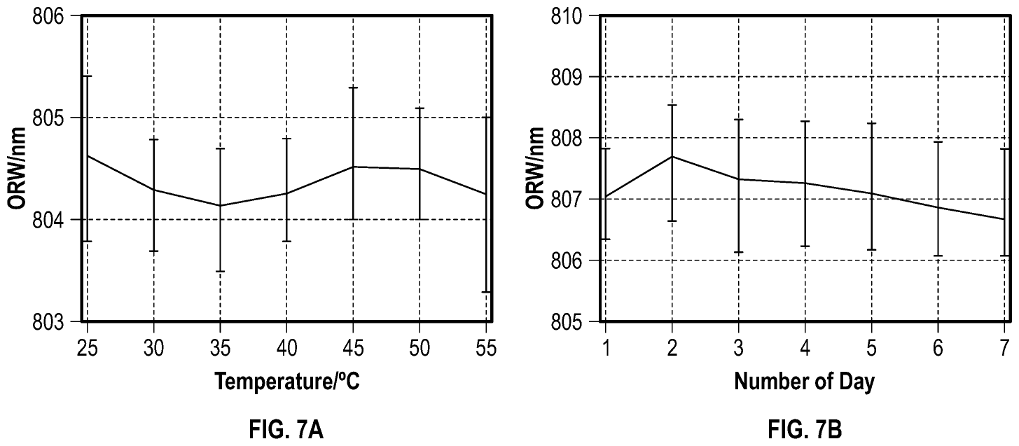

Optical ultrasound transducer arrays in accordance with various example embodiments provide good device stability, and consequently consistent sensing performance for an extended period of operation, both during long-time water immersion of the array, as often needed for acoustic coupling, and over a wide temperature range. For example, in some embodiments, the optical resonance wavelength of the ultrasound transducer array has a relative standard deviation of less than 0.05% over a temperature range from 25° C. to 55° C. and over a period of about a week. This stability is evidenced in FIGS. 7A and 7B.

FIG. 7A is a graph showing the average optical resonance wavelength (ORW) of a set of ultrasound transducer elements of an example array, in accordance with an embodiment, as a function of temperature. The ambient temperature was controlled with a hotplate and monitored with an infrared thermometer. FIG. 7A shows the mean value of the optical resonance frequencies of three elements of the array over a temperature range from 25° C. to 55° C. The measured standard deviation of the optical resonance wavelength is 0.18 nm, which is only 0.022% of the mean value, demonstrating good temperature stability.

FIG. 7B is a graph showing the average optical resonance wavelength of the set of ultrasound transducer elements of FIG. 7A immersed in water over a period. The mean value of the optical resonance wavelength was monitored continuously for seven days. The measured standard deviation over the period of time was 0.33 nm, corresponding to a relative standard deviation of 0.04%, demonstrating good temporal stability.

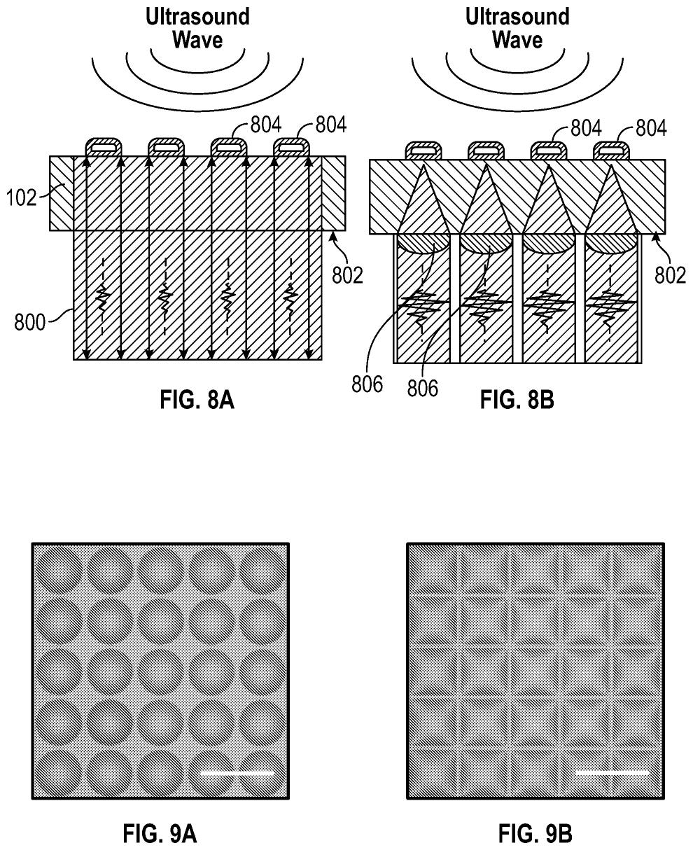

The performance of the disclosed ultrasound transducer arrays can be further improved, in accordance with various embodiments, by integrating an optical interrogation array with the array of Fabry-Pérot-based transducer elements. The optical interrogation array is generally configured to direct light through a back surface of the substrate into and collect reflected light from the individual optical ultrasound transducer elements of the array. Specific implementations include microlens arrays (as illustrated in FIGS. 8A-11) and optical fiber arrays (as shown in FIGS. 12A-12C).

For comparison, FIGS. 8A and 8B are schematic drawings of ultrasound transducer array devices with and without integrated microlenses, respectively, in accordance with various embodiments. In the absence of a microlens or other interrogation array, the array as a whole is interrogated with substantially parallel light, e.g., as provided by a collimated beam 800, as shown in FIG. 8A, or alternatively by an optical fiber bundle abutting the back surface 802 of the substrate. The collimated beam 800 illuminates, through the back surface of the substrate 102, the entire area of the ultrasound transducer array, including the transducer elements 802 (which may be implemented, e.g., as shown in FIGS. 1A and 2A-2F) and any space therebetween. In areas between the transducer elements 802, the optical power is simply lost. To reduce these losses while increasing the modulation efficiency and sensitivity of the acoustic array, an array of microlenses 806 may be formed on the back surface 802 of the substrate 102, as shown in FIG. 8B. Each microlens 806 is aligned with a corresponding one of the optical ultrasound transducer elements 804, and focuses a portion of the light of the interrogating light beam into that transducer element 804. As a result of such focusing, the reflected optical signal received from an ultrasound transducer array with microlenses is generally stronger than that of an ultrasound transducer array without microlenses, as symbolically shown in the figures.

Forming the microlenses 806 involves, in one embodiment, depositing a layer of photoresist (such as, e.g., AZ 9260, available from Merck KGaA) on the back surface of the substrate, and patterning the layer of photoresist to create an array of photoresist islands aligned with the Fabry-Pérot cavities. The photoresist islands are then reshaped into microlenses by thermal reflowing, e.g., in a convection oven. The photoresist islands may be shaped like squares or like circles. Square-shaped islands result, upon reflowing, in three-dimensional microlenses having shapes similar to four-sided pyramids. Circular photoresist islands will be reshaped into microlenses resembling spherical caps or spherical segments in shape. The choice between square and circular cross-sectional shapes may depend on the requirements of the particular application. Square-shaped microlenses can cover the area of the optical ultrasound transducer array with a high filling factor, e.g., of at least 80%, while circular shapes provide for lower optical aberration.

FIGS. 9A and 9B are bottom views of microlens arrays with circular and square cross-sectional shapes, respectively, in accordance with various embodiments.

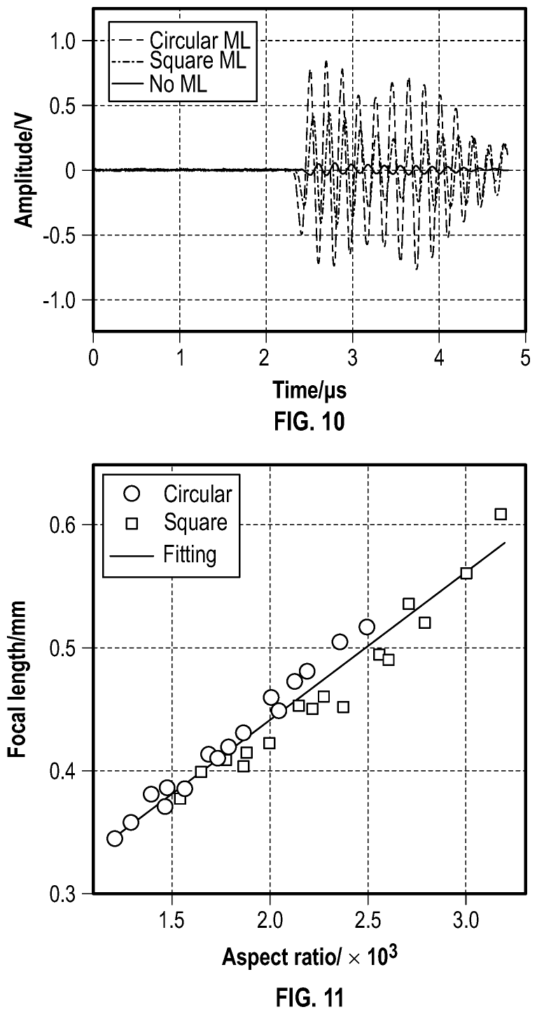

FIG. 10 is a graph of ultrasound signal waveforms measured with various example ultrasound transducer array devices with and without integrated microlenses in accordance with various embodiments. As can be seen, the integration of microlenses significantly increases the signal amplitude, due to improved optical coupling between the interrogating light and the ultrasound transducer elements. Square-shaped microlenses underperform circular ones, which may be the result of their higher aberration.

FIG. 11 is a scatter plot of the focal length of example microlenses as illustrated schematically in FIG. 8B as a function of the aspect ratio—understood as cross-sectional area over thickness—of the photoresist islands. In general, to maximize the illumination efficiency with an array of microlenses, the focal length of the microlenses should be substantially equal to the thickness of the glass substrate. As FIG. 11 shows, control over the focal length can be obtained via adjustments of the aspect ratio. The area of the photoresist islands is largely determined by the spacing between ultrasound transducer elements and the desire to capture as much light as possible. For example, for ultrasound transducer elements having diameters of 70 μm and a pitch of 140 μm, the diameters or side lengths of the photoresist islands from which the microlenses are formed may be between 100 μm and 130 μm (the latter with an increment of 10 μm). Given a certain area, the aspect ratio can be adjusted via the thickness of the photoresist layer. In some embodiments, the photoresist layer is spin-coated onto the back surface of the substrate, allowing the thickness of the layer to be controlled via the spin-coating speed. In some embodiments, an aspect ratio of area over thickness of the photoresist islands is controlled such that the focal length of the microlenses thermal reflow deviates from the thickness of the substrate by less than 2%.

Aside from photoresist patterning and thermal reflow, various other techniques may be employed to create a microlens array for focusing light into the ultrasound transducer elements, improving modulation efficiency. For instance, an array of microlenses may be created, e.g., from polymer, by mold transfer, and then bonded to the back surface of the substrate. Yet another option is to 3D-print microlenses onto the back surface of the substrate. Further, instead of forming three-dimensional microlenses, an array of Fresnel lenses may be created on the back surface of the substrate, e.g., by photolithographically patterning and etching a photoresist layer coated onto the back surface to form the characteristic pattern of rings creating a Fresnel lens.

As an alternative to an array of microlenses, an optical interrogation array configured to transmit light to and from individual ultrasound transducer elements may also be implemented with optical fiber.

FIG. 12A is a schematic drawing of an ultrasound transducer array device 1200 with integrated optical fibers, in accordance with an embodiment. The substrate 1202 on which the ultrasound transducer array is formed is, in this case, an anodically bonded glass-on-silicon substrate, including a glass layer 102 providing the front surface and a silicon layer 1204 providing the back surface of the substrate. The Fabry-Pérot-based ultrasound transducer elements 1206 are formed on the front surface, and thus on the glass side of the substrate, in a manner similar as describe above. To integrate a separate optical fiber with each transducer element, an array of holes is drilled through the silicon layer 1204 of the substrate 1202, ending at the back surface of the glass layer 102. Each hole is aligned with one of the ultrasound transducer elements 1206. Individual optical fibers 1208 are inserted into these holes to illuminate the respective ultrasound transducer elements and capture light reflected form the transducer elements. The optical fibers may be secured in the silicon layer 1204 with an adhesive, such as epoxy 1210.

In some embodiments, the ultrasound transducer array device 1200 is diced into individual sensor units, which may be utilized either as stand-alone sensor probes or re-arranged into a different, e.g., curved, array, as illustrated in FIGS. 12B and 12C.

FIG. 12B is a schematic drawing of a fiber-optic Fabry-Pérot sensor probe 1220 in accordance with an embodiment. This probe corresponds to one die of the diced array device 1200, and accordingly includes a single Fabry-Perot-based optical ultrasound transducer element 1206 and a single associated optical fiber 1208 for optically interrogating the element 1206. Note that, while the probe 1220 can be obtained by subdividing an ultrasound transducer array device 1200 already integrated with optical fiber, it is also possible to first dice an ultrasound transducer array formed on a substrate, and drill the hole through the silicon and insert the optical fiber 1208 thereafter. In principle, it is also possible to manufacture a sensor probe 1220 by itself, but in practice, manufacture of an entire array that is thereafter divided into individual sensor elements or probes will generally be economically advantageous due to economies of scale.

The fiber-optic sensor probe 1220 can be used for various sensing purposes, for example, as an ultrasonic or pressure sensor for endoscopy applications (such as in vivo cardiovascular pressure monitoring). Further, different functional coatings, indicated by coating 1222 in FIG. 12B, can be deposited on the sensor surface to utilize different sensing mechanisms for achieving various sensing functions. For instance, when coated with a porous film like polyimide, or more generally a moisture-absorbing film, the sensor becomes humidity-sensitive owing to water-absorption-induced deformation. A metal coating configures the probe as a temperature sensor because of the bimorph effect, that is, a temperature-induced deformation. The sensor may also serve as a chemical sensor once a thin film absorbing a certain type of chemical is deposited. Polymer film, such as parylene, can be deposited as a damper on the diaphragm, which expands the mechanical resonance frequency bandwidth of the sensor and makes the sensor an ultrasound sensor.

FIG. 12C is a schematic drawing of a curved ultrasound transducer array 1230 constructed from fiber-optic Fabry-Pérot sensor probes in accordance with an embodiment. Here, sensor probes as depicted in FIG. 12B (although not necessarily including the coating 1222) are affixed to a curved support structure, such as, in the depicted example, a hemispheric mold 1232, to form an ultrasound transducer array in three dimensions. Compared with conventional piezoelectric sensor arrays, such a 3D fiber-optic ultrasound transducer array can achieve larger array density with lower system complexity. The 3D fiber-optic ultrasound transducer array can be used for fast volumetric ultrasound and photoacoustic tomography. Suitable molds for specific applications can be made straightforwardly, e.g., by 3D printing.

Creating an ultrasound transducer array from individual sensor probes also facilitate combining ultrasound sensors that differ in their dimensions and accordingly achieve different center frequencies of their respective acoustic frequency spectra in the same array to accommodate both low-frequency and high-frequency ultrasound detection.

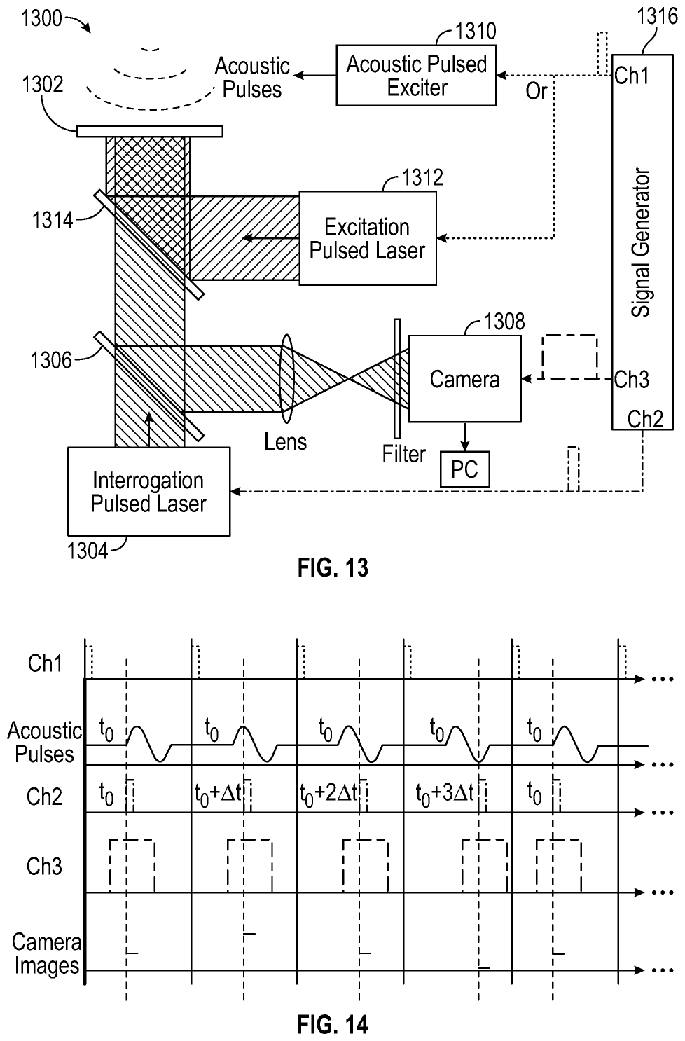

Turning now to the interrogation of ultrasound transducer arrays as described herein in imaging applications, FIG. 13 is a schematic diagram illustrating an example ultrasound or photoacoustic imaging system 1300 utilizing an ultrasound transducer array 1302 in accordance with an embodiment. The transducer array 1302 may be in contact with an acoustic medium, e.g., it may be immersed in water, to achieve acoustic coupling to a target. (In some cases, the target itself is in contact with the transducer array 1302 and serves as the acoustic medium.) The transducer array 1302 is illuminated from the back, through the substrate, by an interrogation pulsed laser 1304 whose operating wavelength is in the vicinity of the optical resonance wavelength of the Fabry-Perot cavities (i.e., within the optical resonance band between the optical resonance wavelength and a wavelength at which the reflectivity has increased almost to the reflectivity outside the resonance). Reflected laser pulses from the ultrasound transducer array 1302 are split off by a polarization beam splitter 1306 and captured by a camera 1308 sensitive at the operating wavelength as a 2D image, whose brightness is proportional to the intensity and pulse duration of the interrogation pulsed laser 1304.

To generate acoustic waves in the target, an acoustic pulsed exciter 1310 may be used. Alternatively, for photoacoustic imaging, an excitation pulsed laser 1312 may be used to induce acoustic waves in the target. In the depicted configuration, the excitation laser 1312 operates in a wavelength range to which the ultrasound transducer array 1302, including its substrate, is transparent, which enables transmitting the excitation pulses through the transducer array 1302 and, conveniently, placing the excitation pulsed laser 1312 on the on the same side of the transducer array 1302 as the interrogation pulses laser 1304. The laser pulses from the excitation pulsed laser 1312 and from the interrogation pulsed laser 1304 may be combined along the same path (e.g., along the optical axis of the optical ultrasound transducer array 1302) by a dichroic mirror 1314. A three-channel signal generator 1316 generates trigger signal to coordinate the relative timing of excitation and interrogation laser pulses and signal capture by the camera 1308.

The system 1300 facilitates the fully parallelized optical read-out of the ultrasound transducer array. In various embodiments, it utilizes a Nd:YAG laser, which operates at 532 nm, as the interrogation pulsed laser 1304, and a conventional CMOS or CCD camera 1308 to image the reflected light. Nd:YAG lasers are well-suited for ultrasound and photoacoustic imaging due to their ability to generate short pulses, e.g., of 10 ns or less, at relatively high pulse energies (e.g., of 0.1 mJ or more) without the need for the addition of amplifiers, which facilitates high sampling rates (as needed to resolve high-frequency ultrasound signals) at good signal levels. In addition, Nd:YAG lasers are cheap as compared with many pulsed laser systems at other operating wavelengths. For example, at 1550 nm, a complex and expensive system including, among other things, an electro-optic modulator and fiber amplifier is applied to convert continuous-wave output from a tunable laser into 30 ns pulsed output, while still suffering from low light intensity. Also, image capture at 1550 nm may rely on a special and expensive InGaAs camera, whose frame rate and sensitivity may not be optimal. In previous systems, such drawbacks were inevitable because the interrogation system had to be adapted in its operating wavelength to the fixed resonance wavelength of the optical ultrasound transducer array. The surface-micromachined Fabry-Perot-based ultrasound transducer arrays described herein, on the other hand, can be flexibly adjusted to the operating wavelength of the interrogation system (e.g., to a wavelength around 532 nm, meaning sufficiently close to 532 nm that the 532 nm interrogating light falls within the resonance band), facilitating the use of the cheaper and better-performing Nd:YAG laser and cheaper cameras.

FIG. 14 is a conceptual signal-flow diagram illustrating an aspect of the operation of the photoacoustic imaging system of FIG. 13. A first trigger signal (Channel 1) is used to periodically trigger the acoustic pulsed exciter 1310 or excitation pulsed laser 1312. The generated acoustic pulses, which almost linearly modulate the optical reflectivity of the ultrasound transducer array 1302, have a constant delay t₀ from Channel 1. A second trigger signal (Channel 2) is used to trigger the probe laser pulses of the interrogation pulsed laser 1304 to interrogate the acoustically modulated reflectivity of the optical ultrasound transducer array 1302. The trigger of Channel 2 is programmed with an increasing delay from that of Channel 1, which is t₀+(N−1)×Δt at the N^{th} period (N=acoustic pulse duration/Δt). For example, with N=4, the acoustic pulse is sampled at four time delays within four periods. A third trigger signal (Channel 3) triggers image capture by the camera 1308. Channel 3 is synchronous with Channel 2, except that it has a slightly earlier starting time (which may be regarded a fixed offset) and wider duration to ensure that the short laser pulses can be captured by the camera. The brightness in the camera images in each period is used to reconstruct the acoustic pulse. With a smaller sampling interval Δt, the sampling frequency can be as high as twice of the central frequency of the acoustic pulse for optimal signal reconstruction.

Although one of the benefits of the described optical ultrasound transducer arrays is their amenability to parallel read-out, other read-out schemes are not thereby excluded. For example, it is possible to read out an optical transducer array by scanning an interrogating beam with multiple focal spots or a focused line over the transducer array to increase the readout speed.

Having described various aspects and features of the inventive subject matter, the following numbered examples are provided as illustrative embodiments:
1. A surface-micromachined optical ultrasound transducer array device includes: a substrate; a planar first partially optically reflective layer disposed on a front surface of the substrate; a second partially optically reflective layer disposed over the first partially optically reflective layer; and an array of Fabry-Pérot cavities defined between the first and second partially reflective layers, the Fabry-Pérot cavities being bounded at the bottom by the first partially optically reflective layer and at the top and sides by respective flexible diaphragms and side walls formed by the second partially optically reflective layer, the array of Fabry-Pérot cavities and respective flexible diaphragms together forming an array of optical ultrasound transducer elements.
2. In the device of example 1, the first and second partially reflective layers are distributed Bragg reflectors each formed by a stack of alternating material layers.
3. In the device of example 2, a resonance wavelength of the Fabry-Pérot cavities is within a reflection band of the distributed Bragg reflectors.
4. In the device of example 2 or example 3, the alternating material layers comprise oxide and nitride layers.
5. The device of any of examples 1-4 further includes a sealing layer disposed over the second partially optically reflective layer.
6. In the device of example 5, the sealing layer is made of or includes a low-temperature oxide.
7. The device of example 5 or example 6 further includes a damping layer disposed over the sealing layer.
8. In the device of example 7, a thickness of the damping layer is between about 1 μm and about 50 μm.
9. In the device of example 8, an acoustic frequency spectrum of the optical ultrasound transducer elements is characterized by a bandwidth greater than 80% of a center frequency of the acoustic frequency spectrum.
10. In the device of any of examples 1-9, a pitch of the array of optical ultrasound transducer elements is less than half of an acoustic wavelength at a center frequency of an acoustic frequency spectrum of the optical ultrasound transducer elements.
11. In the device of any of examples 1-10, the Fabry-Pérot cavities have cavity lengths deviating by no more than 10 nm from an average cavity length of the array of Fabry-Pérot cavities.
12. In the device of any of examples 1-11, the optical ultrasound transducer elements of the array each have dimensions of less than 100 μm, and at least 90% of the optical ultrasound transducer elements of the array have associated optical resonance wavelengths deviating by no more than 5 nm from an average optical resonance wavelength of the array of optical ultrasound transducer elements.
13. In the device of any of examples 1-12, an optical resonance wavelength of the array of ultrasound transducer elements has a relative standard deviation of less than 0.05% over a temperature range from 25° C. to 55° C.
14. In the device of any of examples 1-13, dimensions of the optical ultrasound transducer elements are less than 200 μm.
15. The device of any of examples 1-14 further includes: an optical interrogation array configured to direct light through a back surface of the substrate into and collect reflected light from the individual optical ultrasound transducer elements of the array.
16. In the device of example 15, the optical interrogation array is or includes a microlens array disposed on the back surface of the substrate, the array including microlenses each aligned with one of the optical ultrasound transducer elements.
17. In the device of example 16, the microlenses have circular cross-sectional shapes.
18. In the device of example 16, the microlenses have square cross-sectional shapes and cover an area of the optical interrogation array with a filling factor of at least 80%.
19. In the device of any of examples 16-18, the focal lengths of the microlenses deviates from a thickness of the substrate by less than 2%.
20. In the device of any of examples 16-19, the microlenses are Fresnel lenses.
21. In the device of example 15, the optical interrogation array includes an array of optical fibers extending from the back surface of the substrate at least partially through the substrate, each optical fiber being aligned with one of the optical ultrasound transducer elements.
22. The device of any of examples 1-20 further includes an imaging optical fiber bundle configured to direct light through a back surface of the substrate into and collect reflected light from the array of optical ultrasound transducer elements to image the array.
23. A method of manufacturing an optical ultrasound transducer array includes: depositing a planar first partially optically reflective layer on a front surface of a substrate; depositing a sacrificial layer on the first partially optically reflective layer; patterning the sacrificial layer to form an array of islands of sacrificial material; depositing a second partially optically reflective layer over the array of islands on the first partially optically reflective layer; removing the sacrificial material to form an array of Fabry-Pérot cavities between the first and second partially optically reflective layers; and depositing a sealing layer over the second partially optically reflective layer to vacuum-seal the Fabry-Pérot cavities. The Fabry-Pérot cavities are bounded at the bottom by the first partially optically reflective layer and at the top and sides by respective flexible diaphragms and side walls formed by the second partially optically reflective layer, and the array of Fabry-Pérot cavities and respective flexible diaphragms together form an array of optical ultrasound transducer elements.
24. In the method of example 23, depositing the planar first partially optically reflective layer and the second partially optically reflective layer each include depositing multiple pairs of oxide and nitride layers collectively forming a distributed Bragg reflector.
25. In the method of example 24, deposition of the pairs of oxide and nitride layers is preceded by deposition of dummy oxide and nitride layers and measurement of refractive indexes of the dummy oxide and nitride layers, and the thicknesses of the oxide and nitride layers of each pair are selected based on the measured refractive indexes such that the oxide and nitride layers each have an optical length equal to one quarter of a target center wavelength of the distributed Bragg reflector.
26. The method of any of examples 23-25 further includes: depositing a damping layer over the sealing layer.
27. In the method of example 26, a cavity length of the Fabry-Pérot cavities is tuned in part by adjusting at least one of a thickness of the sealing layer or a thickness of the damping layer.
28. In the method of example 27, a thickness of the damping layer is selected at least in part based on a target bandwidth of an acoustic frequency spectrum of the optical ultrasound transducer elements.
29. In the method of any of examples 23-28, dimensions of the optical ultrasound transducer elements are selected at least in part based on a target center frequency of an acoustic frequency spectrum of the optical ultrasound transducer elements.
30. In the method of any of examples 23-29, the Fabry-Pérot cavities are vacuum-sealed inside a vacuum oven at an ambient pressure selected based on a target internal pressure inside the Fabry-Pérot cavities corresponding to a target bandwidth of the acoustic frequency spectrum of the optical ultrasound transducer elements.
31. The method of any of examples 23-30 further includes: forming a first electrode beneath the first partially optically reflective layer and a second electrode above the second partially optically reflective layer; and actively controlling a cavity length of the Fabry-Pérot cavities by application of a voltage across the Fabry-Pérot cavities between the first and second electrodes.
32. The method of any of examples 23-30 further includes: depositing, over the second partially optically reflective layer, a coating that has a thermal coefficient of expansion that differs by at least 10% from a thermal coefficient of expansion of the second partially optically reflective layer, the coating and flexible diaphragms formed by the second partially optically reflective layer together forming bimorph structures; and actively controlling a cavity length of the Fabry-Pérot cavities by adjusting a temperature of the bimorph structure.
33. In the method of any of examples 23-32, the substrate is a glass substrate, and the method further includes: forming an array of microlenses on a back surface of the substrate, each of the microlenses aligned with one of the optical ultrasound transducer elements.
34. In the method of example 33, forming the array of microlenses includes: depositing a layer of photoresist on the back surface of the substrate; patterning the layer of photoresist to create an array of photoresist islands aligned with the ultrasound transducer elements; and reshaping the photoresist islands into microlenses by thermal reflow.
35. In the method of example 34, an aspect ratio of area over thickness of the photoresist islands is controlled such that a focal length of the microlenses resulting from the thermal reflow deviates from a thickness of the substrate by less than 2%.
36. In the method of example 35, the layer of photoresist is spin-coated onto the back surface of the substrate at a spin-coating speed selected based on a target thickness of the layer of photoresist.
37. In the method of example 33, forming the array of microlenses includes: forming an array of polymer microlenses by mold transfer; and bonding the array of polymer microlenses to the back surface of the substrate.
38. In the method of example 33, forming the array of microlenses includes: 3D-printing the array of microlenses onto the back surface of the substrate.
39. In the method of example 33, forming the array of microlenses includes: depositing a layer of photoresist on the back surface of the substrate; and patterning the layer of photoresist to form Fresnel lenses.
40. In the method of any of examples 23-32, the substrate is an anodically bonded glass-on-silicon substrate and the Fabry-Pérot cavities are formed on a glass side of the substrate, and the method further includes: drilling an array of holes into the silicon side of the substrate; and inserting optical fibers through the holes, the optical fibers each being aligned with one of the Fabry-Pérot cavities.
41. The method of example 40 further includes: dicing the array of optical ultrasound transducer elements formed on the substrate into a plurality of individual ultrasound transducer elements; integrating the individual ultrasound transducer elements with respective optical fibers to form a plurality of individual ultrasound sensor probes; and affixing the plurality of individual ultrasound sensor probes to a curved support structure to form a curved optical ultrasound transducer array.
42. An optically interrogated ultrasound imaging system includes: an optical ultrasound transducer array comprising, formed on a front surface of a substrate, a plurality of Fabry-Pérot cavities having a shared optical resonance band around 532 nm and bounded in part by flexible diaphragms, the optical resonance band shifting responsive to acoustic waves impinging on the flexible diaphragms due to changes in a cavity length of the Fabry-Pérot cavities; a Nd-YAG interrogation pulsed laser having an operating wavelength within the optical resonance band of the Fabry-Pérot cavities between an optical resonance wavelength associated with the optical resonance band and a wavelength of highest reflectivity associated with the optical resonance band, arranged to illuminate the optical ultrasound transducer array through a back surface of the substrate; and a camera sensitive to light reflected in a range comprising the operating wavelength, arranged to capture light reflected from the Fabry-Pérot cavities through the back surface of the substrate.
43. In the ultrasound imaging system of example 42, the camera is a CCD or CMOS camera.
44. In the ultrasound imaging system of example 42 or example 43, the interrogation pulsed laser is configured to generate laser pulses of less than 10 ns in length.
45. In the ultrasound imaging system of any of examples 42-44, the interrogation pulsed laser is configured to generate laser pulses with energies of more than 0.1 mJ.
46. The ultrasound imaging system of any of examples 42-45 further includes: an excitation pulsed laser having an operating wavelength outside the optical resonance band of the Fabry-Pérot cavities, arranged to emit excitation light pulses through the optical ultrasound transducer array in a direction from a back surface of the substrate to the front surface of the substrate to excite ultrasound waves in an acoustic medium in contact with the ultrasound transducer array for photoacoustic imaging; and a dichroic mirror to combine light from the interrogation pulsed laser and the excitation pulsed laser along an optical axis through the optical ultrasound transducer array.
47. The ultrasound imaging system of example 46 further includes an electronic signal generator configured to generate trigger signals for the excitation pulsed laser, the interrogation pulsed laser, and the camera, wherein the trigger signal for the interrogation pulsed laser has a fixed offset relative to the trigger signal for the camera, and wherein the trigger signals for the interrogation pulsed laser and the camera have a periodically varying offset relative to the trigger signal for the excitation pulsed laser so as to temporally resolve acoustic pulses measured by the ultrasound transducer array upon excitation of the ultrasound waves in the acoustic medium.
48. One or more sensor probes each include: a substrate; formed on the substrate, a Fabry-Pérot sensor; and an optical fiber extending through a back surface of the substrate and at least partially through the substrate to carry light for the interferometric interrogation of the Fabry-Pérot sensor. The Fabry-Pérot sensor includes a planar first partially optically reflective layer disposed on a front surface of the substrate, a second partially optically reflective layer disposed over the first partially optically reflective layer, and a Fabry-Pérot cavity defined between the first and second partially reflective layers, the cavity being bounded at the bottom by the first partially optically reflective layer and at the top and sides by respective flexible diaphragms and side walls formed by the second partially optically reflective layer.
49. In the one or more sensor probes of example 48, the substrate is a glass-on-silicon substrate and the front surface is on a glass layer of the substrate, and the optical fiber extends through a silicon layer of the substrate to a back surface of the glass layer.
50. In the one or more sensor probes of example 48 or example 49, the first and second partially reflective layers are distributed Bragg reflectors each formed by a stack of alternating material layers.
51. In the one or more sensor probes of any of examples 48-50, each sensor probe further includes a sealing layer disposed over the second partially optically reflective layer.
52. In the one or more sensor probes of example 48, each sensor probe further includes a functional coating disposed over or above the second partially optically reflective layer. The functional coating selected from the group consisting of: a moisture-absorbing film configuring the sensor probe as a humidity sensor; a metal film configuring the sensor probe as a temperature sensor; a chemically absorptive film configuring the sensor probe as a chemical sensor; and a polymer film configuring the sensor probe as a damped ultrasound sensor.
53. The one or more sensor probes of any of examples 48-52 include a plurality of sensor probes arranged in an array.
54. The plurality of sensor probes of example 53 are arranged in an array on a curved surface.
55. In the plurality of sensor probes of example 53 or example 54, the sensor probes are configured as ultrasound sensors.
56. In the plurality of sensor probes of example 55, the ultrasound sensors differ in their dimensions to achieve different center frequencies of their respective acoustic frequency spectra.
57. A method of manufacturing one or more sensor probes includes: depositing a planar first partially optically reflective layer on a front surface of a substrate; depositing a sacrificial layer on the planar first partially optically reflective layer; patterning the sacrificial layer to form one or more islands of sacrificial material; depositing a second partially optically reflective layer disposed over the one or more islands on the first partially optically reflective layer; removing the sacrificial material to form one or more Fabry-Pérot cavities between the first and second partially optically reflective layers, the Fabry-Pérot cavities being bounded at the bottom by the first partially optically reflective layer and at the top and sides by respective flexible diaphragms and side walls formed by the second partially optically reflective layer; vacuum-sealing the one or more Fabry-Pérot cavities; and integrating each of the one or more Fabry-Pérot cavities with an optical fiber for optically interrogating the Fabry-Pérot cavity.
58. The method of example 57 further includes: applying a functional coating over or above the second partially optically reflective layer of the one or more Fabry-Pérot cavities, the functional coating selected from the group consisting of a moisture-absorbing film, a metal film, a chemically absorptive film, or a polymer film.
59. The method of example 57 or example 58, wherein the sacrificial layer is patterned to form an array of multiple islands of sacrificial material, which results, upon removal of the sacrificial material, in an array of multiple Fabry-Pérot cavities. The method further includes dicing the array of Fabry-Pérot cavities formed on the substrate to create multiple sensor probes.

Although embodiments have been described with reference to specific example embodiments, it will be evident that various modifications and changes may be made to these embodiments without departing from the broader scope of the invention. Accordingly, the specification and drawings are to be regarded in an illustrative rather than a restrictive sense.

1. A surface-micromachined optical ultrasound transducer array device comprising:
a substrate;
a planar first partially optically reflective layer disposed on a front surface of the substrate;
a second partially optically reflective layer disposed over the first partially optically reflective layer; and
an array of Fabry-Pérot cavities defined between the first and second partially reflective layers, the Fabry-Pérot cavities being bounded at its bottom by the first partially optically reflective layer and its a top and sides by respective flexible diaphragms and side walls formed by the second partially optically reflective layer, the array of Fabry-Pérot cavities and respective flexible diaphragms together forming an array of optical ultrasound transducer elements.

2. The device of claim 1, wherein the first and second partially optically reflective layers are distributed Bragg reflectors each formed by a stack of alternating material layers.

3. The device of claim 2, wherein a resonance wavelength of the Fabry-Pérot cavities is within a reflection band of the distributed Bragg reflectors.

4. The device of claim 2, wherein the alternating material layers comprise oxide and nitride layers.

5. The device of claim 1, further comprising a sealing layer disposed over the second partially optically reflective layer.

6. The device of claim 5, wherein the sealing layer comprises a low-temperature oxide.

7. The device of claim 5, further comprising a damping layer disposed over the sealing layer.

8. (canceled)

9. (canceled)

10. The device of claim 1, wherein a pitch of the array of optical ultrasound transducer elements is less than half of an acoustic wavelength at a center frequency of an acoustic frequency spectrum of the optical ultrasound transducer elements.

11. The device of claim 1, wherein the Fabry-Pérot cavities have cavity lengths deviating by no more than 10 nm from an average cavity length of the array of Fabry-Pérot cavities.

12. The device of claim 1, wherein the optical ultrasound transducer elements of the array each have dimensions of less than 100 μm, and wherein at least 90% of the optical ultrasound transducer elements of the array have associated optical resonance wavelengths deviating by no more than 5 nm from an average optical resonance wavelength of the array of optical ultrasound transducer elements.

13. The device of claim 1, wherein an optical resonance wavelength of the array of ultrasound transducer elements has a relative standard deviation of less than 0.05% over a temperature range from 25° C. to 55° C.

14. The device of claim 1, wherein dimensions of the optical ultrasound transducer elements are less than 200 μm.

15. The device of claim 1, further comprising:
an optical interrogation array configured to direct light through a back surface of the substrate into and collect reflected light from the optical ultrasound transducer elements of the array.

16. The device of claim 15, wherein the optical interrogation array comprises a microlens array disposed on the back surface of the substrate and comprising microlenses each aligned with one of the optical ultrasound transducer elements.

17. The device of claim 16, wherein the microlenses have circular cross-sectional shapes.

18. The device of claim 16 wherein the microlenses have square cross-sectional shapes and cover an area of the optical interrogation array with a filling factor of at least 80%.

19. The device of claim 16, wherein focal lengths of the microlenses deviate from a thickness of the substrate by less than 2%.

20. The device of claim 16, wherein the microlenses are Fresnel lenses.

21. The device of claim 15, wherein the optical interrogation array comprises an array of optical fibers extending from the back surface of the substrate at least partially through the substrate, each optical fiber being aligned with one of the optical ultrasound transducer elements.

22. The device of claim 1, further comprising:
an imaging optical fiber bundle configured to direct light through a back surface of the substrate into and collect reflected light from the array of optical ultrasound transducer elements to image the array.

23-59. (canceled)
